(19) United States
(12) Patent Application Publication
(10) Pub. No.: US 2026/0249107 A1
(43) Pub. Date: Aug. 27, 2026

(54) ULTRASONIC CERVICAL VAGUS NERVE STIMULATOR

(71) Applicant: NeurGear, Inc., Rochester, NY (US)

(72) Inventors: Stanley Ward Stephenson, Spencerport, NY (US); Jonathan Taylor Hacker, Rochester, NY (US); Andrew Robert Slegaitis, Forestport, NY (US)

(21) Appl. No.: 19/547,484

(22) Filed: Feb. 23, 2026

Related U.S. Application Data
(60) Provisional application No. 63/834,115, filed on Feb. 24, 2025.

(51) Int. Cl. A61N 7/00

(52) U.S. Cl. CPC A61N 7/00; A61N 2007/0026

(57) ABSTRACT

Apparatus for stimulating a cervical vagus nerve including a neck band attachable to a human neck, a therapy module attached to said neck band wherein said neck band positions said therapy module over a cervical vagus nerve, an ultrasound transducer within said therapy module, and a driver circuit configured to excite said ultrasound transducer to apply ultrasonic energy to said cervical vagus nerve. A force-limiting mechanism including a spring-loaded subframe prevents damage to blood vessels adjacent to said cervical vagus nerve. The apparatus operates at low voltages of 5 volts or less suitable for portable battery-powered operation and provides pulsed ultrasonic energy for therapeutic stimulation.

### CROSS REFERENCE TO RELATED APPLICATIONS

This application is a nonprovisional conversion of and claims priority to US Provisional Application 63/834,115 filed Feb. 24, 2025, the entirety of which is incorporated by reference herein.

### FIELD OF DISCLOSURE

The present disclosure relates to the use of ultrasound to stimulate the cervical vagus nerve.

### BACKGROUND

The center of human consciousness is the brain. The brain is composed of nerves that electrochemically transition between states. Nerve cells store information in the soma of cells and transmit signals using cell axons. The brain sends and receives information to and senses the state of the other areas of the body. It excretes hormones, triggers reflexes, and moves the body in response to external stimulation. Nerves connect the brain to all parts of the body.

The vagus nerve, also known as “the wanderer”, extends from the brain to carry signals between your brain, heart and digestive system. They're a key part of the parasympathetic nervous system. The major branches of the vagus nerve, referred to as the cervical carotid nerve Vc, lie adjacent to the carotid arteries in the sides of the neck. The cervical carotid arteries Vc contain 75% of your parasympathetic nervous system's nerve fibers.

The Mayo Clinic and Cleveland hospital websites describe implanted Vagus nerve stimulation (VNS), which uses electrical impulses to stimulate your left vagus nerve using electrodes around the left cervical vagus nerve. Healthcare providers implant a small device in the chest, under skin. A wire runs under the skin connecting the device and nerve neural cuff. The device sends mild, painless electrical signals through the left vagus nerve to your brain. These impulses calm down irregular electrical activity in your brain. The U.S. Food and Drug Administration (FDA) has approved VNS to treat epilepsy and depression that doesn't respond to standard therapies. It's also being investigated for the treatment of: Cluster headaches, Inflammatory bowel disease (IBD), Pain, Post-traumatic stress disorder (PTSD), Rheumatoid arthritis.

Several companies offer external electrical stimulation devices for the cervical vagus nerves under the brand names Electrocore and Pulsetto. A device contains a pair of electrical contacts that are pressed to the surface of the skin over one or more cervical vagus nerves and electrical pulses are applied to stimulate the cervical vagus nerves. A current carrying electrode gel is required to transmit electrical pulses through the skin. Such devices require increasing amounts of painful excitation to achieve effect. The electrodes on the device connect across the surface of the skin and the vagus nerve lies below the electrodes. Alternating current is used, which is typically conducted across the surface of the skin, which limits the degree of stimulation of the cervical vagus nerve.

Two branches of the vagus nerve, the auricular vagus nerves Va, lie under the human ears, pinna P. The applicants have developed a device, the Zenbud, which excites the auricular vagus nerves Va using ultrasonic energy. An ultrasound probe is pressed against the external surface of the ear and ultrasonic energy is pulsed over the auricular vagus nerve Va to stimulate nerve Va and induce calm and wellness. The device works on a small branch of the vagus nerve, and stimulation is limited.

Multiple hospital websites describe the use of ultrasound imaging systems to examine the carotid arteries. An ultrasonic probe is placed on the surface of the skin over the carotid artery and ultrasonic radiation irradiates the carotid artery and reflected radiation is used to construct a cross section of the carotid artery to find possible buildup that could lead to a stroke. Such systems are painless and benign in application.

The vagus nerve lies on the sides of the neck, adjacent to several blood vessels. The jugular vein lies near the surface of the neck and can be collapsed by light amounts of pressure. The carotid artery lies deeper in the neck, and the vagus nerve lies near the carotid artery. Excessive pressure will collapse the carotid artery, causing unconsciousness. The trachea lies in the front of the neck and is more resistant to pressures, but can be collapsed by high amounts of pressure.

Effective ultrasound application near cervical blood vessels requires light contact pressure to prevent collapse. Effective pressure for that should be well below the strangulation force to the human neck. The New York City Mayors Office of Domestic Abuse lists excessive strangulation forces. Only 11 lbs. of pressure placed on both carotid arteries for 10 seconds is necessary to cause unconsciousness, 4.4 lbs. of pressure placed on the jugular for 10 seconds is necessary to cause unconsciousness, and 33 lbs. of pressure on the trachea is required to completely close it off. The area over which those forces are applied is not defined by the guideline, and force over a given surface area cannot be determined. Contact force on the neck over the carotid arteries for ultrasonic probes should be well below that value to prevent collapse of the jugular vein, and carotid artery. The pressure applied to an ultrasound probe should be significantly less than pressures described in the guidelines. Other conduits, such as the trachea can withstand higher forces, but any device contact should be well below the guideline forces.

It would be useful to provide stimulate the vagus nerve to induce wellness. It would be useful is the stimulation method was noninvasive and painless. It would be useful if the stimulation was excitation was applied to the major cervical vagus nerve. The stimulation should be performed in a manner that does not harm a user.

### SUMMARY

It is an object of the present disclosure to stimulate the cervical vagus nerve using ultrasonic radiation while preventing damage to adjacent blood vessels. With this object in mind, the present disclosure provides apparatus for brain neurotherapy including a neck band attachable to a human neck, a therapy module attached to the neck band, wherein the neck band positions the therapy module over a cervical vagus nerve, an ultrasound transducer within the therapy module, and a driver circuit configured to excite the ultrasound transducer to apply ultrasonic energy to the cervical vagus nerve.

In one aspect, the apparatus includes a second therapy module positioned to apply ultrasonic energy to a second cervical vagus nerve on an opposite side of the neck, enabling bilateral stimulation of both cervical vagus nerves simultaneously.

In another aspect, the neck band comprises a right arm and a left arm connected by a hinge, with a torsional spring applying force to urge the arms toward each other. An elastic pad is disposed over the hinge to provide a reaction force to the back of the neck. Alternatively, the neck band comprises one or more elastic bands that permit compliance of the therapy module to the surface of the neck over the cervical vagus nerve.

In another aspect, the therapy module is connected to the neck band through a pivot that permits a surface of the therapy module to conform to varying shapes of the neck.

In another aspect, a piezo element is mounted on a subframe held by the therapy module, the subframe capable of movement toward the cervical vagus nerve relative to the therapy module. A spring urges the subframe towards the cervical vagus nerve at a force below a damaging level to blood vessels adjacent to the cervical vagus nerve. The spring is compressed greater than the range of motion of the subframe, so that the urging force changes little when the subframe is depressed. In a preferred embodiment, the spring provides a force of approximately 20 grams on a subframe having a diameter of approximately 14 millimeters.

In another aspect, the driver circuit operates at a frequency in the range of 20 kHz to 100 MHz, with the frequency preferably being approximately one megahertz to limit energy flow to the areas around the cervical vagus nerve.

In another aspect, the therapy module carries drive electronics for the ultrasound transducer, wherein the drive electronics comprise a Hartley oscillator circuit. A battery power source provides 5 volts or less to the driver circuit, enabling portable operation. The battery power source may be a rechargeable battery.

In another aspect, the present disclosure provides a cervical vagus nerve stimulation system comprising a portable housing, at least one piezoelectric transducer configured to contact skin over a cervical vagus nerve, a driver circuit operating at 10 volts or less to excite the piezoelectric transducer, and a controller for providing pulsed ultrasonic energy to the cervical vagus nerve. The portable housing is configured for attachment to a neck band.

The controller activates the piezoelectric transducer for a first time period and deactivates the piezoelectric transducer for a second time period. The first time period is approximately 30 milliseconds and the second time period is approximately 30 milliseconds. The pulsed ultrasonic energy is provided for a therapeutic period of approximately 5 minutes.

The piezoelectric transducer has a resonant frequency of approximately one megahertz. The driver circuit comprises inductors and capacitors configured to resonate at the resonant frequency of the piezoelectric transducer. The system may further comprise two piezoelectric transducers for bilateral stimulation of cervical vagus nerves, and a user interface for selecting therapy parameters.

In another aspect, the present disclosure provides a wearable ultrasonic therapy device for cervical vagus nerve stimulation comprising a frame, a button movably held within the frame and guided by the frame to move toward a cervical vagus nerve, a piezo element mounted on the button, a spring urging the button outward from the frame with a force below a damaging level to blood vessels in the neck, and a neck band for securing the frame to a human neck over the cervical vagus nerve.

The piezo element is covered with epoxy. The spring is compressed significantly greater than the possible motion of the button so that the urging force changes little when the button is depressed. The button includes a detent preventing the button from exiting the frame. A cover is attached to the frame providing a reaction surface for the spring. The button projects outward from the frame to conform to variations in the surface of the neck.

These objects are given only by way of illustrative example, and such objects may be exemplary of one or more embodiments of the disclosure. Other desirable objectives and advantages inherently achieved by the disclosed disclosure may occur or become apparent to those skilled in the art. The invention is defined by the appended claims.

### BRIEF DESCRIPTION OF DRAWINGS

The foregoing and other objects, features, and advantages of the invention will be apparent from the following more particular description of the embodiments of the invention, as illustrated in the accompanying drawings. The elements of the drawings are not necessarily to scale relative to each other.

FIG. 1 is a sectional view through the human neck showing the cervical vagus nerve.

FIG. 2 is an isometric view of a human head having apparatus in accordance with the present invention.

FIG. 3 is a front view of the therapeutic device 20 attached to the human neck.

FIG. 4 is an isometric upper rear view of the invention.

FIG. 5 is a top view of a therapeutic device with the cover removed.

FIG. 6 is a side sectional view of the frame of FIG. 5.

FIG. 7 is sectional view showing the relationship of the therapeutic device on human anatomy.

FIG. 8 is a view of another embodiment of the invention.

FIG. 9 is a block diagram of the electrical circuit for the apparatus.

FIG. 10 is a schematic of a piezo driver circuit in the invention.

FIG. 11 is a plot of the output of piezo driver 44.

FIG. 12 is a diagram of energy pulses that are generated by controller 50 of FIG. 9.

PARTS LIST

| Reference | Element |
|---|---|
| 10 | human head |
| 12 | human brain |
| 14 | human neck |
| 15 | jugular vein |
| 16 | carotid artery |
| 18 | trachea |
| 20 | therapy module |
| 30 | frame |
| 32 | button |
| 34 | spring |
| 36 | cover |
| 38 | cover hinge |
| 40 | piezo crystal |
| 44 | piezo driver |
| 48 | power source |
| 50 | controller |
| 52 | user interface |
| 60 | neck band |
| 62 | right arm |
| 64 | left arm |
| 66 | neck band pivot hinge |
| 68 | neck pad |
| 70 | drive electronics |
| 80 | elastic band |
| Vc | Cervical Vagus nerve |

### DETAILED DESCRIPTION

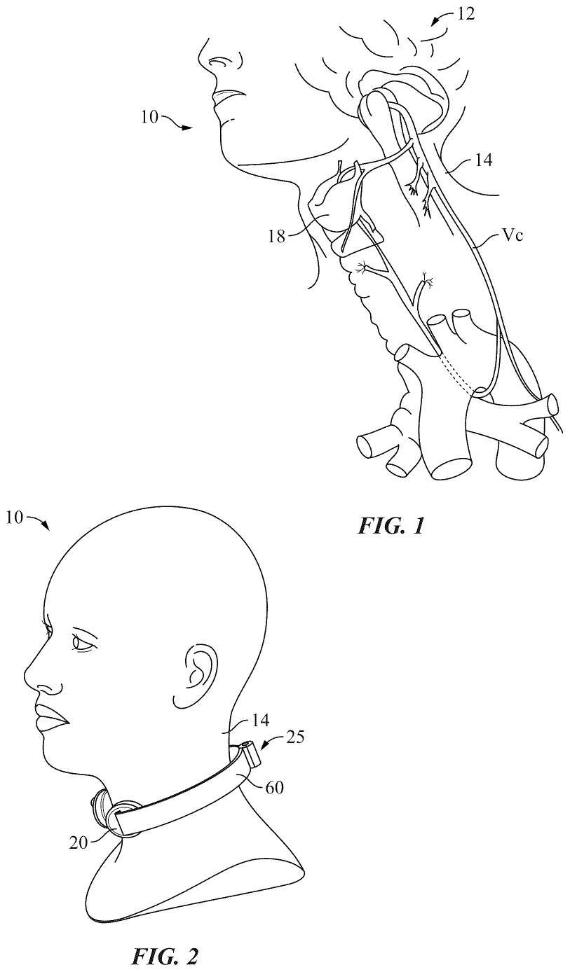

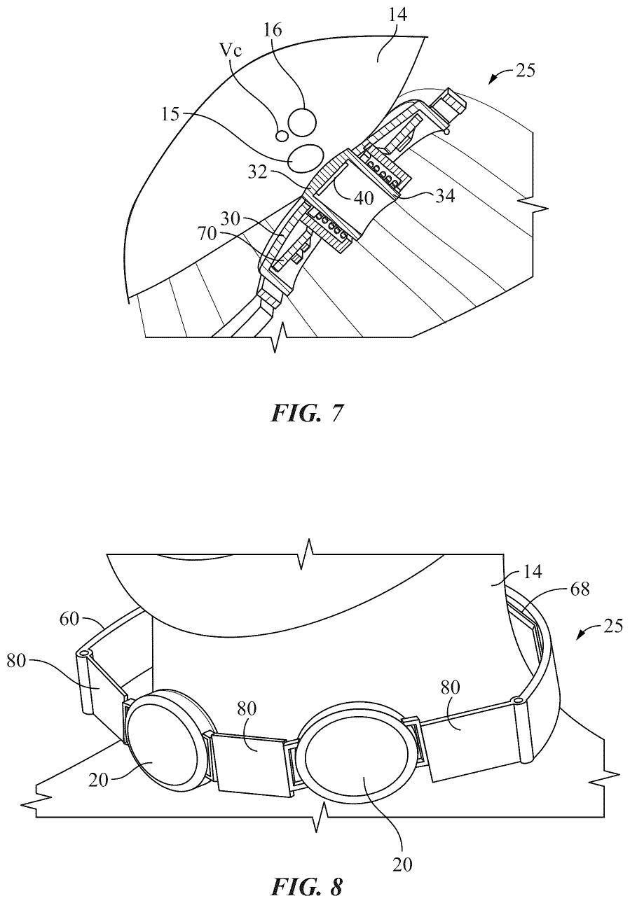

FIG. 1 is a sectional view through the human neck showing the cervical vagus nerve. A human head 10 is supported by the human neck 14. Two cervical vagus nerves Vc lie adjacent to the trachea 18 and carotid artery on either side of human neck 14. Cervical vagus nerves Vc connect the human brain 12 to organs in the viscera, including the heart and digestive system. The jugular veins 15 lie above the carotid artery 16 and cervical vagus nerve, as shown in FIG. 7, closer to the surface of human neck 14.

FIG. 2 is an isometric view of a human head 10 having apparatus in accordance with the present invention. A therapeutic device 25 supporting a therapy module 20 is secured to human neck 14 by a neck band 60 and positions therapy module 20 over cervical vagus nerve Vc. Neck band 60 conforms generally to the curved surface of a human neck 14, and can fully or partially encircle the human neck 14. Neck band 60 carries one or two therapy modules 20 which emit ultrasonic radiation. Frame 30 of therapy module 20 has a surface in contact with human neck 14 over cervical vagus nerves Vc.

Neck band 60 can be made of a flexible material to urge frame 30 against neck 14. Neck band 60 can be made of one or more polymeric materials, such as silicone rubber, spandex, neoprene or latex, that can be knitted, woven or braided. Forming sequential small folds in a flexible thread provides stretching with little tensile force. Neck band 60 can be formed of multiple parts of rigid and flexible materials to secure one or more therapy modules 20 to human neck 14. Neck band 60 should hold frame 30 to neck 14 below a force that would damage the carotid artery 16 or jugular vein 15.

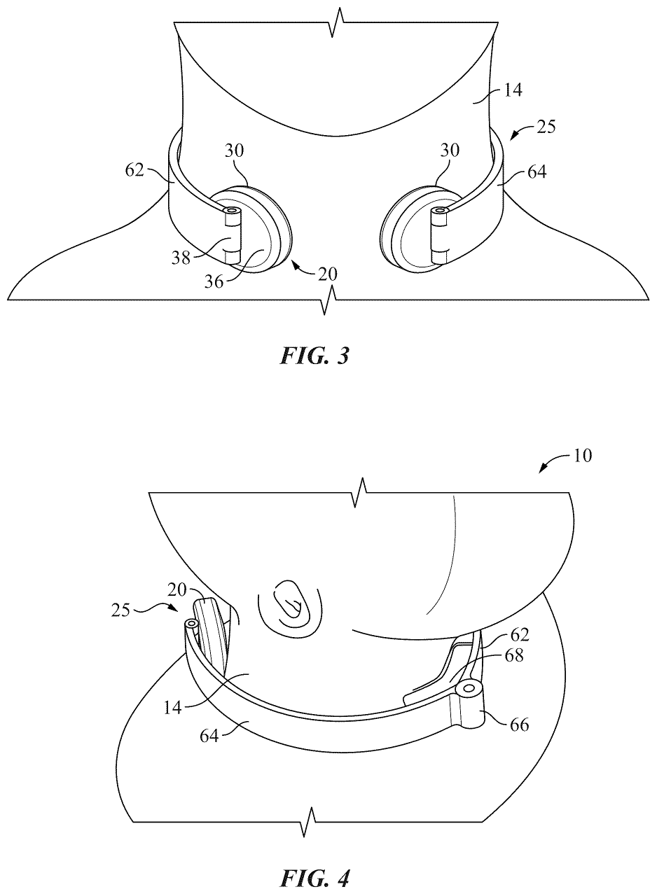

FIG. 3 is a front view of the therapeutic device 25 attached to the human neck. In this embodiment, neck band 60 consists of a right arm 62 and a left arm 64 that press therapy modules 20 against both cervical vagus nerves Vc. Cover 36 is attached to frame 30 and protects internal components generating ultrasound. Covers 36 over frames 30 connect frame 30 to neck band 60 through a pivoting cover hinge 38 that permits a surface of frames 30 to conform to varying shapes of human neck 14.

FIG. 4 is an isometric upper rear view of the therapeutic device 25. Neck band 60 encircles human neck 14 and is secured to frame 30. Right arm 62 and left arm 64 are connected by neck band pivot hinge 66 that permits the two arms to move relative to each other. Forcing means such as a torsional spring, not shown, applies a torsional force to the two arms to press the arms toward each other. A neck pad 68 is disposed over neck hinge to provide a reaction force to the back of human neck 14. Alternatively, the arms and hinge can be replaced by an elastic band that presses therapy modules 20 against cervical vagus nerves Vc.

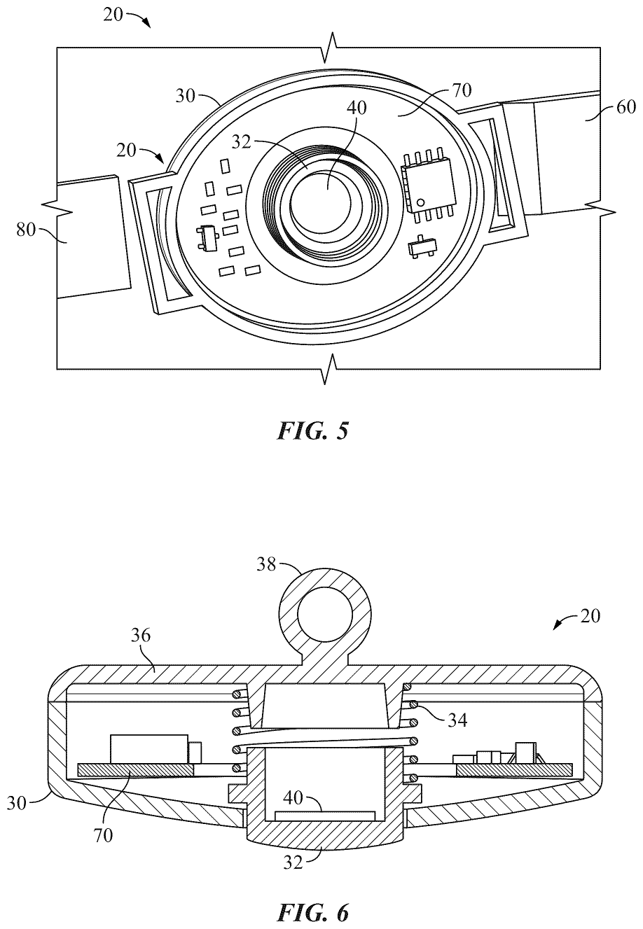

FIG. 5 is a top view of a therapy module 20 with the cover 36 removed. Therapy module 20 is held in place by neck band 60. Drive electronics 70 drives piezo element 40 through wires, not shown. Piezo element 40 is mounted on button 32. Button 32 is guided by frame 30 to move towards cervical vagus nerve Vc. Circuit board 70 has a board that holds components and conforms to the interior of frame 30.

FIG. 6 is a side sectional view of the frame of FIG. 5. Piezo element 40 is mounted inside button 32. Piezo element 40 could be attached to the surface of button 32 facing human neck 14 and be covered with epoxy. Buton 32 is held by frame 30 and permits free movement of button 32 towards vagus nerve Vc. Detail in button 32 prevents it from exiting frame 30 when being forced from frame 30. Spring 34 defines the contact force over cervical vagus nerve Vc, Spring 34 is configured to provide an urging force well below a damaging force to blood vessels in the neck. Cover 36 provides a reaction surface for spring 34 to apply force on button 32. Spring 34 can be a wound metal wire of length and wire diameter to provide a force to drive button 32 towards carotid artery 16 without damage. Spring 34 is compressed significantly greater than the possible motion of button 32 so that the urging force changes little when button 32 is depressed into frame 30. Spring 34 can be a cantilever spring formed in the plastic parts of other materials, such as an elastomeric pad.

FIG. 7 is sectional view showing the relationship of the therapeutic device 25 on human anatomy. Frame 30 is held onto the surface of human neck 14 by neck band 60. Button 32 is held in frame 30 and free to press against neck 14. Frame 30 orients button 32 over cervical vagus nerve Vc which lies adjacent to carotid artery 16 and under jugular vein 15. Spring 34 urges button into human neck 14.

The force applied by spring 34 is less than the force that would collapse carotid artery 16 or jugular vein 15. Button 32 projects out from frame 30 and conform to variations in the surface of neck 14 over cervical vagus nerve Vc. The force applied by spring 34 is sufficient to provide light contact to the surface of the skin over neck 14 to permit transmission of ultrasound energy from piezo element 40 but not damage blood vessels. In the preferred embodiment of the invention, the force urging button 32 outward is 20 grams on a 14 millimeter in diameter button 32.

FIG. 8 is a view of another embodiment of the invention. Therapy modules 20 are held against the neck by one or more elastic bands 80. In this embodiment, three separate elastic bands 80 are used to attach two therapy modules 20 to neck band 60. Neck band 60 in this case is a unitary loop that encircles the back of neck 14 and has attachment points to elastic bands 80. Neck band 60 can include neck pads 68 that provide soft contact surfaces on either side of the back of neck 14. Elastic bands 80 permit therapy modules 20 to rotate to conform to the surfaces of neck 14 over the cervical vagus nerves Vc. A single elastic band 80 can be threaded through loops in therapy modules 20. In the single band case, therapy modules 20 can slide along the elastic band 80 to position therapy modules 20 over the cervical vagus nerves Vc.

Elastic bands 80 with therapy modules 20 can be selectively attached to neck band 60. The attaching means can be permanent on one side and releasable on the opposite side to permit the assembly to be wrapped around human neck 14. Force limiting apparatus can be included in addition to elastic bands 80 to minimize force applied to blood vessels in human neck 14 over the force applied by the stretching of elastic bands 80.

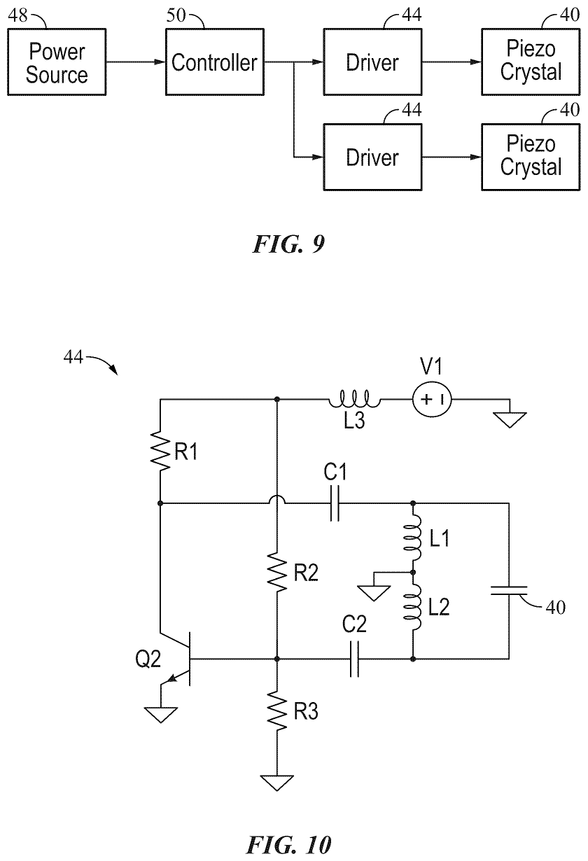

FIG. 9 is a block diagram of the electrical circuit for the apparatus. Drive electronics 70 can contain controller 50, user interface 52, and two piezo driver circuits 44 that power piezo crystals 40. Energy for drive electronics 70 is supplied by power source 48 which another embodiment is a rechargeable battery. Power source 48 can be embedded in one or more frames 30 or be attached to therapy modules 20. Power source 48 can be a wall connection of standard design. Drive electronics 70 can be a single circuit board or distributed across multiple boards housed within therapy modules 20. User interface 52 permits selection of therapy parameters including pulse timing and therapeutic period duration.

FIG. 10 is a schematic of a piezo driver circuit of the therapeutic device 25. Piezo driver 44 is a Hartley circuit which has inductors L1 and L2 and capacitors C1 and C2 that resonate at the resonant frequency of piezo element 40. Piezo element 40 is designed to resonate in the 300 kHz to 5 MHz range to transmit energy to cervical vagus nerve Vc. Frequencies around one-megahertz limits energy flow to the areas around cervical vagus nerve Vc. Piezo element 40 has a very low effect on the Hartley circuit and follows the voltage oscillations between the poles.

A first pole of resonance, between L1 and C1, can store energy as a high positive voltage. An opposing negative voltage occurs at a second pole between L2 and C2. The voltage potential between pole 1 and pole 2 causes stored energy to flow from the first pole to the second pole. Inductors L1 and L2 develop a magnetic field that drives the charge into the second pole. The voltage across poles one and two is reversed by the movement of energy from pole 1 to pole 2. Resistances in the circuit and the parasitic load form piezo element 40 causes energy loss during resonance, and the circuit will decay rapidly without the addition of energy.

Resistors R2 and R3 provide a reference voltage define an ON voltage for transistor Q2. The ON voltage for transistor Q2 is selected to add energy to the resonant circuit at a time that maximizes the peak voltage in the resonant circuit. The maximized voltage maximizes the energy emitted by piezo element 40. R1 limits the current that is supplied to piezo driver 44. Inductor L3 provides a secondary resonance to piezo driver 44 which improves peak voltage in the resonance circuit and increase power output from piezo element 40.

Piezo driver 44 operates efficiently using a minimum number of components in a compact space compared to other commercial products. Piezo driver circuit is designed to operate at low voltages that are readily available, such as 5 volts from a USB cable or 3.3 or 6 volts from batteries. The preferred embodiment requires that the components be selected to operate with the supplied voltage. Commercial ultrasound units typically use high voltages and power for ultrasound generation, which prevents untethered portability of such as battery 48. A circuit in accordance with the present invention creates a small portable stimulation device.

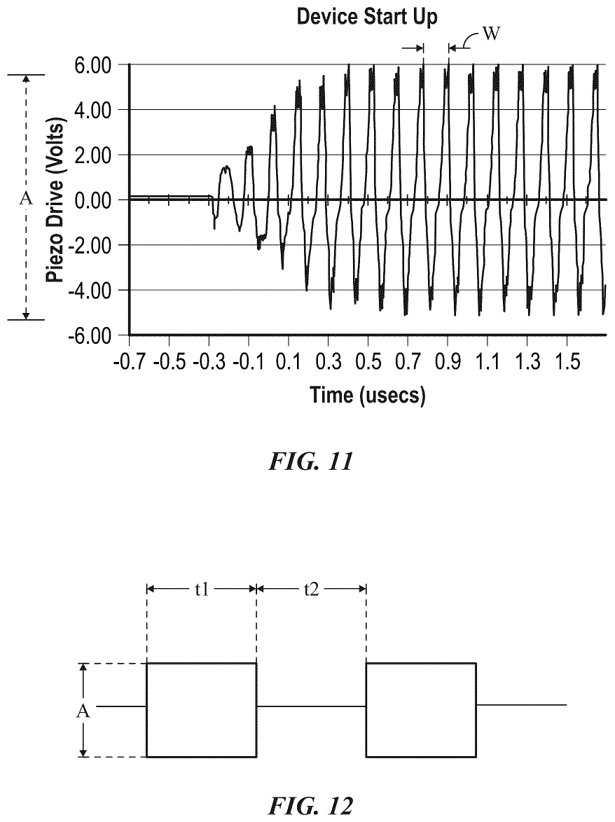

FIG. 11 is a plot of the output of piezo driver 44. A device was constructed in accordance with the electrical design on FIG. 9 and FIG. 10. The circuit was designed to resonate at 8 Megahertz at 5 volts with an attached 8-megahertz crystal. Piezo driver 44 applied voltage to piezo element 40 to create mechanical vibrations above human hearing range, known as ultrasound. The circuit resonated to the maximum applied 5 volts. The circuit came to resonance after 6 cycles. The circuit was detached from power and decayed in the same time.

FIG. 12 is a diagram of energy pulses that are generated by controller 50, of FIG. 9. Controller 50 turns on piezo driver 44 for a first time period t1 to excite vagus nerve Vc at power level P. Controller 50 turns off piezo driver 44 for a second time period t2. Vagus nerve Vc responds to the applied energy biochemically during time period t1. When the applied power is removed during time period t2, vagus nerve Vc resets biochemically. The periodic energy provides therapeutic signals to the brain, imparting relaxation. In one case, piezo element 40 is activated for approximately 30 milliseconds and then deactivated for approximately 30 milliseconds. The process continues for a therapeutic period of time, such as approximately 5 minutes.

An invention has been disclosed that orients ultrasonic transducers over the cervical vagus nerves. Various mechanical embodiments of the invention exist to perform that function. The apparatus includes force limiting means to prevent damage to a user from the collapse of blood vessels. The apparatus provides painless therapeutic excitation of to the major vagus nerves.

The invention has been described in detail, and may have been described with particular reference to a suitable or presently preferred embodiment, but it will be understood that variations and modifications can be affected within the spirit and scope of the invention. For example, therapeutic device 25 can be incorporated for operation within a closed loop system that includes sensors for detecting conditions or patterns in subject physiology. The presently disclosed embodiments are therefore considered in all respects to be illustrative and not restrictive. The scope of the invention is indicated by the appended claims, and all changes that come within the meaning and range of equivalents thereof are intended to be embraced therein.

We claim:

1. Apparatus for stimulating a cervical vagus nerve comprising:
a) a neck band attachable to a human neck;
b) a therapy module attached to said neck band, wherein said neck band positions said therapy module over a cervical vagus nerve;
c) an ultrasound transducer within said therapy module; and
d) a driver circuit configured to excite said ultrasound transducer to apply ultrasonic energy to said cervical vagus nerve.

2. The apparatus of claim 1 further comprising a second therapy module positioned to apply ultrasonic energy to a second cervical vagus nerve on an opposite side of said neck.

3. The apparatus of claim 1 wherein:
a) a piezo element is mounted on a subframe held by said therapy module, said subframe capable of movement toward said cervical vagus nerve relative to said therapy module; and
b) a spring configured to urge said subframe towards said cervical vagus nerve at a force below a damaging level to blood vessels adjacent to said cervical vagus nerve.

4. The apparatus of claim 3 wherein said spring is compressed greater than a range of motion of said subframe.

5. The apparatus of claim 3 wherein said spring provides a force of approximately 20 grams on a subframe having a diameter of approximately 14 millimeters.

6. The apparatus of claim 1 wherein said driver circuit operates at a frequency in a range of 20 kHz to 100 MHz.

7. The apparatus of claim 6 wherein said frequency is approximately one megahertz.

8. The apparatus of claim 1 wherein said neck band comprises a right arm and a left arm connected by a hinge, and a torsional spring applying force to urge said arms toward each other.

9. The apparatus of claim 8 further comprising an elastic pad disposed over said hinge to provide a reaction force to a back of said neck.

10. The apparatus of claim 1 wherein said neck band comprises one or more elastic bands that permit compliance of said therapy module to a surface of said neck over said cervical vagus nerve.

11. The apparatus of claim 1 wherein said therapy module is connected to said neck band through a pivot that permits a surface of said therapy module to conform to varying shapes of said neck.

12. The apparatus of claim 1 wherein said therapy module carries drive electronics for said ultrasound transducer.

13. The apparatus of claim 12 wherein said drive electronics comprise a Hartley oscillator circuit.

14. The apparatus of claim 1 further comprising a battery power source providing 5 volts or less to said driver circuit.

15. A cervical vagus nerve stimulation system comprising:
a) a portable housing;
b) at least one piezoelectric transducer configured to contact skin over a cervical vagus nerve;
c) a driver circuit operating at 10 volts or less to excite said piezoelectric transducer; and
d) a controller for providing pulsed ultrasonic energy to said cervical vagus nerve.

16. The system of claim 15 wherein said controller activates said piezoelectric transducer for a first time period and deactivates said piezoelectric transducer for a second time period.

17. The system of claim 16 wherein said first time period is approximately 30 milliseconds and said second time period is approximately 30 milliseconds.

18. The system of claim 15 wherein said pulsed ultrasonic energy is provided for a therapeutic period of approximately 5 minutes.

19. The system of claim 15 wherein said piezoelectric transducer has a resonant frequency of approximately one megahertz.

20. The system of claim 15 wherein said driver circuit comprises inductors and capacitors configured to resonate at a resonant frequency of said piezoelectric transducer.

21. The system of claim 15 further comprising a rechargeable battery.

22. The system of claim 15 further comprising two piezoelectric transducers for bilateral stimulation of cervical vagus nerves.

23. The system of claim 15 further comprising a user interface for selecting therapy parameters.

24. The system of claim 15 wherein said portable housing is configured for attachment to a neck band.

25. A wearable ultrasonic therapy device for cervical vagus nerve stimulation comprising:
a) a frame;
b) a button movably held within said frame and guided by said frame to move toward a cervical vagus nerve;
c) a piezo element mounted on said button;
d) a spring urging said button outward from said frame with a force below a damaging level to blood vessels in a neck; and
e) a neck band for securing said frame to a human neck over said cervical vagus nerve.

26. The device of claim 25 wherein said piezo element is covered with epoxy.

27. The device of claim 25 wherein said spring is compressed significantly greater than a possible motion of said button so that an urging force changes little when said button is depressed.

28. The device of claim 25 wherein said button includes a detent preventing said button from exiting said frame.

29. The device of claim 25 further comprising a cover attached to said frame providing a reaction surface for said spring.

30. The device of claim 25 wherein said button projects outward from said frame to conform to variations in a surface of said neck.
